(19) United States
(12) Patent Application Publication
(10) Pub. No.: US 2015/0083126 A1
(43) Pub. Date: Mar. 26, 2015

(54) Breathing Circuit Humidification System

(71) Applicant: David D. Rogers, Quakertown, PA (US)

(72) Inventor: David D. Rogers, Quakertown, PA (US)

(73) Assignee: Draeger Medical Systems, Inc., Andover, MA (US)

(21) Appl. No.: 14/397,456

(22) PCT Filed: Apr. 27, 2012
(86) PCT No.: PCT/US12/35644

§ 371 Date: Oct. 27, 2014

(51) Int. Cl. A61M 16/16; A61M 16/04; A61M 16/20; A61M 16/10; A61M 16/12; A61M 16/00; A61M 16/06

(52) U.S. Cl. CPC A61M 16/161; A61M 16/0003; A61M 16/0057; A61M 16/04; A61M 16/06; A61M 16/109; A61M 16/12; A61M 16/205; A61M 2016/0027; A61M 2202/0208; A61M 2202/0225; A61M 2202/0266; A61M 2202/025; A61M 2205/3327; A61M 2205/3368; A61M 2205/50
USPC 128/203.14; 128/203.27

(57) ABSTRACT

A breathing circuit humidification system (200) is described that includes a humidification system (102) and a patient breathing circuit (201). The humidification system includes at least a moisture water source (209), a flash evaporator (211) and a moisture exchanger. Water is selectively introduced from the water source to the flash evaporator. The flash evaporator includes a heating element that evaporates the received water to form water vapor. The water vapor is sent to the moisture exchanger. The moisture exchanger receives gas from a gas source. The received gas is humidified with the water vapor received from the flash evaporator. The moisture exchanger sends the humidified gas to the patient breathing circuit. The patient breathing circuit provides, in a controlled manner, the humidified gas to a patient (106). Related methods, apparatus, systems, techniques and articles are also described.

### TECHNICAL FIELD

The subject matter described herein relates to a breathing circuit humidification system that humidifies gas and provides the humidified gas to a patient in a controlled manner.

### BACKGROUND

In respiration support systems, there may be a requirement to humidify breathing air as the breathing air is provided to a patient. The breathing air is conventionally humidified using a humidification system that is powered by an alternating current voltage source. Further, such a conventional humidification system is usually bulky, stationary, large in size, and no portion of the humidification system is disposable.

### SUMMARY

The current subject matter describes a breathing circuit humidification system that includes a humidification system and a breathing circuit. The humidification system can humidify gas and provide the humidified gas to a patient breathing circuit. The patient breathing circuit can further provide, in a controlled manner, the humidified gas to a patient. The humidification system can include at least a moisture water source, a flash evaporator and a moisture exchanger. Water is selectively introduced from the water source to the flash evaporator. The flash evaporator can include a heating element that can evaporate the received water to form water vapor. The water vapor can be sent to the moisture exchanger. The moisture exchanger can receive gas from a gas source. The received gas can be humidified with the water vapor received from the flash evaporator. The moisture exchanger can send the humidified gas to the patient breathing circuit. The patient breathing circuit can provide, in a controlled manner, the humidified gas to a patient. Related methods, apparatus, systems, techniques and articles are also described.

In one aspect, a system is described that can include a water source, a flash evaporator and a moisture exchanger. The flash evaporator can selectively receive water from the water source, and evaporates at least a portion of the received water to form water vapor. The moisture exchanger can receive the water vapor from the flash evaporator, receive gas from a gas source, and humidify the received gas with the received water vapor. The humidified gas can be transported to a patient breathing circuit.

The system can further include temperature sensors and humidity sensors. The temperature sensors can measure temperature of the humidified gas in the patient breathing circuit. The humidity sensors can measure humidity of the humidified gas in the patient breathing circuit.

The system can further include a control unit coupled to the temperature sensors and the humidity sensors. The control unit can allow for selective introduction of water from the water source into the flash evaporator. The selective introduction can be based on the humidity measured by the humidity sensors.

The system can include a solenoid valve and a control unit. The solenoid valve can selectively open to selectively introduce water from the water source and to the flash evaporator. The selective introduction of water can cause the selective receiving of the water by the flash evaporator. The control unit can control, based on an amount of moisture present in the patient breathing circuit, the selectively opening of the solenoid valve.

The water source can provide a continuous supply of water that can selectively pass through the solenoid valve. The continuous supply of water can be provided using a gravitational force.

The system further can include a temperature sensor and a humidity sensor. The temperature sensor can determine a temperature of the humidified gas. The humidity sensor can determine the amount of moisture present in the patient breathing circuit.

The flash evaporator can include a heating element that can evaporate the water to form the water vapor. The heating element can temporarily stop evaporating the water when the temperature determined by the temperature sensor exceeds a predetermined threshold.

An amount of the water vapor and an amount of the gas that are received at the moisture exchanger can be based on the amount of moisture determined by the humidity sensor.

The system can further include an overflow collector that can collect excess water vapor that is not mixed with the received gas to form humidified gas.

The system can further include a direct current voltage source that can provide power to the flash evaporator, the moisture exchanger, and the control unit.

The system can include an alternating current voltage source that can provide power to the flash evaporator, the moisture exchanger, and the control unit.

The flash evaporator can include a structure having a tee (T) shape. The structure can have a first gap, a second gap, and a third gap. The flash evaporator can include a heating element that evaporates water to form water vapor. The heating element can be coupled to the tee (T) shaped structure so as to seal the first gap.

The water from the water source can be received by the flash evaporator from the second gap. The third gap can be sealed by a fixed solenoid valve.

The heating element can slide along an inside surface of a portion of the tee (T) shaped structure.

The heating element can slide along an outside surface of a portion of the tee (T) shaped structure.

The moisture exchanger can include a semi-permeable membrane tube that can receive the water vapor from the flash evaporator. The semi-permeable membrane tube can allow a first portion of the received water vapor to escape out of the semi-permeable membrane tube while prohibiting a second portion of the received water vapor that can condense to form liquid water to escape out of the semi-permeable membrane tube.

The water vapor that escapes out of the semi-permeable membrane tube can be mixed with the gas received at the moisture exchanger to form humidified gas that can be sent to the patient breathing circuit.

The patient breathing circuit and the moisture exchanger can be disposable while the water source and the flash evaporator can be reusable.

The received gas can include a breathing gas that can include at least one of oxygen, carbon dioxide, nitrogen, helium, and neon.

The patient breathing circuit can include an inspiratory portion and an expiratory portion. The inspiratory portion can facilitate provision of humidified gas to a patient. The expiratory portion can facilitate removal, from the patient breathing circuit, of gas exhaled by the patient. The inspiratory portion can be enclosed by a carbon-fiber enclosure.

In another aspect, water vapor can be formed by evaporation of water selectively received at a flash evaporator from a water source. The water vapor can be received from the flash evaporator and at a first opening in a semi-permeable tube within a moisture exchanger apparatus. A portion of the water vapor can escape out of the semi-permeable tube. At a second opening of the moisture exchanger apparatus and from a gas source, gas can be received. In a region outside the semi-permeable tube, the escaped water vapor can be mixed with the gas to form humidified gas. From a third opening of the moisture exchanger apparatus, the humidified gas can be sent to a patient breathing circuit.

From a fourth opening of the moisture exchanger apparatus, excess water vapor that condenses to form liquid can be sent to an overflow collector. The liquefied water vapor can be prohibited to escape from the semi-permeable tube.

A portion of the flash evaporator can be enclosed by a carbon-fiber housing.

In one aspect, an apparatus is described that can include a gas inlet, a water vapor inlet, a humidified gas outlet and an overflow outlet. The gas inlet can receive gas from a gas source. The water vapor inlet can receive water vapor in a semi-permeable tube coupled with the water vapor inlet. A first portion of the received water vapor can permeate through the semi-permeable tube to mix with the received gas to form humidified gas. A second portion of the received water vapor can be prevented from permeating through the semi-permeable tube. The humidified gas outlet can send the humidified gas to a patient breathing circuit. The overflow outlet can send the second portion of the received water vapor to an overflow collector.

The second portion of the received water vapor can condense to form liquid water. The semi-permeable tube can prevent permeation of the liquid water.

The liquid water collected in the overflow collector can be reused by a water source that can selectively provide water to an evaporator that can evaporate water to form the received water vapor. The received gas can be oxygen.

In another aspect, an apparatus is described that can include a fitting, an inspiratory section, and an expiratory section. The fitting can include a proximal portion and a distal portion. The proximal portion can be connected to an inhalation device of a patient. The inspiratory section can be coupled to the distal portion of the fitting and can receive humidified gas from a humidification system. At least a portion of the inspiratory section being enclosed by a heating enclosure. The expiratory portion can be coupled to the distal portion of the fitting and can remove gas exhaled by the patient.

The inspiratory section can be an inspiratory tube, and the expiratory portion can be an expiratory tube.

The heating enclosure can comprise a carbon-fiber sleeve that can envelope at least a portion of the inspiratory tube. The heating enclosure can be coupled to a controller that can selectively apply current to the carbon-fiber sleeve so as to selectively heat the inspiratory section. The controller can selectively apply current to the carbon-fiber sleeve to selectively heat the humidified gas within the inspiratory section.

The fitting is a unitary Y-shaped fitting. The humidified gas is prevented from entering the expiratory section. The exhaled gas is prevented from entering the inspiratory section. The humidified gas can be oxygen that can be humidified in accordance with a medical recommendation for the patient.

The inhalation device can be a mask that can be placed on a face of the patient. In another variation, the inhalation device can be an endotracheal tube that can be inserted within a trachea of the patient.

The subject matter described herein provides many advantages, some of which are noted below. For example, the humidification system and patient breathing circuit can be hygienic, low cost, light, portable, and small (for example, can fit in a 2 feet×2 feet space) in size.

Further, some components of the humidification system and breathing circuit can include, by a direct or indirect contact with a patient, some portion of saliva and/or air exhaled by the patient. Those components of the humidification system and breathing circuit can be disposable, thereby advantageously preventing transmission of infection, microorganisms, and/or the like. So, a cross-contamination between various patients can be prevented. Moreover, disposability of some components can prevent contamination caused by sticking/harboring of microorganisms on wet surfaces of those components. Thus, the disposability of some components allows a hygienic provision of humidified gas to a patient.

Furthermore, some other components of the humidification system can be reusable. A reuse of such components can be advantageously cost efficient, thereby saving cost for a patient.

Moreover, the humidification system can be powered by a direct current source that can advantageously allow a portable implementation of the humidification system, which in turn allows the humidification system to be implemented in an automobile, an ambulance, an aircraft, a ship, train, neonatal transport, and the like.

Further, in the humidification system, the heating source (that is, a heating element in the flash evaporator) can be advantageously separate from the received gas such as dry oxygen. Such a separation can prevent the dry breathing air from heating up and undesirably/unnecessarily expanding by heat from the heating source.

Furthermore, the flash evaporator can be enclosed/wrapped by an insulator with low thermal conductivity. The use of such an insulator can advantageously ensure safety of a patient and/or clinician using the humidification system, as they can be protected from the heat generated internally within the breathing circuit humidification system.

Further, the insulator-enclosing can be further enclosed by an aluminum housing or carbon fiber housing. The aluminum housing or carbon fiber housing can advantageously allow maintaining the temperature of the flash evaporator below a threshold value of temperature.

Furthermore, an inspiratory portion of the breathing circuit can facilitate provision of humidified gas to a patient. The inspiratory portion can be enclosed by a heating element (for example, a carbon-fiber enclosure) that can have an electrical-resistance property, due to which humidified gas within the inspiratory portion can be heated, as required. It can be advantageous to enclose the inspiratory portion by a heating element rather than including the heater within the inspiratory portion, as a combination of the heating element within the inspiratrory portion and some gases can cause accidents. Thus, enclosing the inspiratory portion with the heating element can advantageously ensure safety of the patient.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 illustrates a breathing circuit humidification system;

FIG. 2 illustrates a breathing circuit humidification system;

FIG. 3 illustrates a portion of a breathing circuit of the breathing circuit humidification system;

FIG. 4 illustrates another orientation of the portion of a breathing circuit of the breathing circuit humidification system;

FIG. 5 illustrates a breathing circuit humidification system;

FIG. 6 illustrates a flash evaporator;

FIG. 7 illustrates a moisture exchanger;

FIG. 8 illustrates a carbon fiber sleeve enclosing an inspiratory portion of the breathing circuit; and

FIG. 9 is a process-flow diagram illustrating humidification of gas using the humidification system.

Like reference symbols in the various drawings indicate like elements.

### DETAILED DESCRIPTION

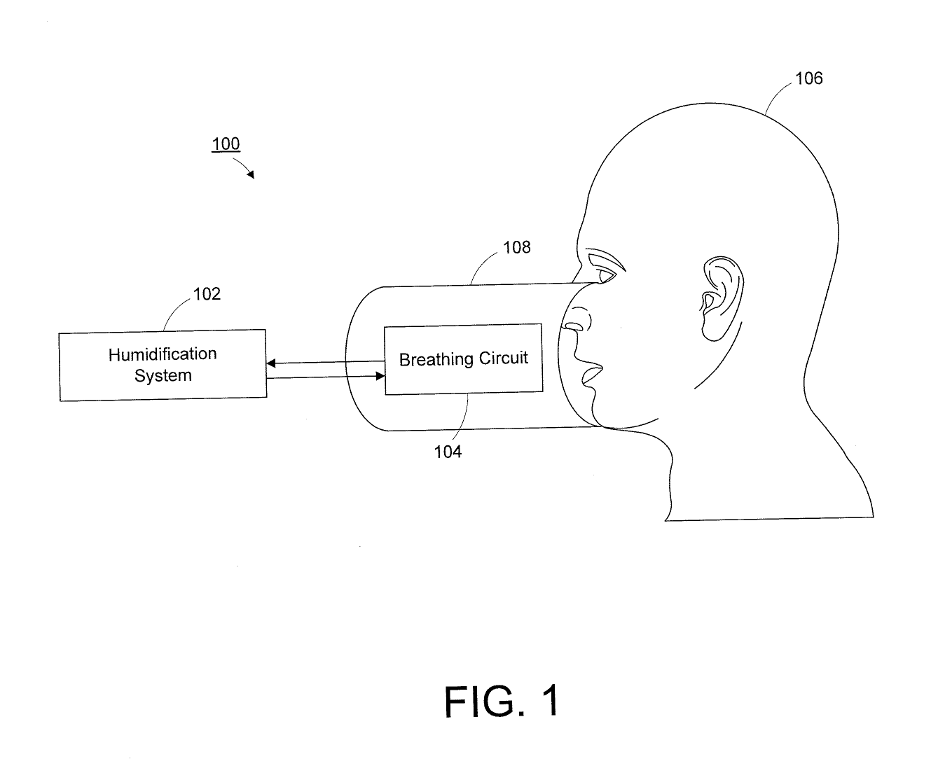

FIG. 1 illustrates a breathing circuit humidification system 100. The breathing circuit humidification system 100 includes a humidification system 102 coupled to a breathing circuit 104. The humidification system 102 can provide humidified gas to a breathing circuit 104 for an individual (for example, patient) 106. The humidified gas can include an appropriate predetermined quantity of water vapor mixed with corresponding predetermined quantity of gas. The gas that is humidified can be dry breathing air, such as dry oxygen.

The breathing circuit 104 can be implemented using an inhalation device, such as a mask 108 provided to the patient 106. The patient 106 can be one of a neonatal patient (for example, a baby), a child, an adult, an animal, and the like. The mask 108 can be temporarily implemented over at least one of a nose and a mouth of the patient 106. In another implementation, some portion of the breathing circuit 104 can be inserted into a trachea of the patient 106, which can be an intubated-neonate.

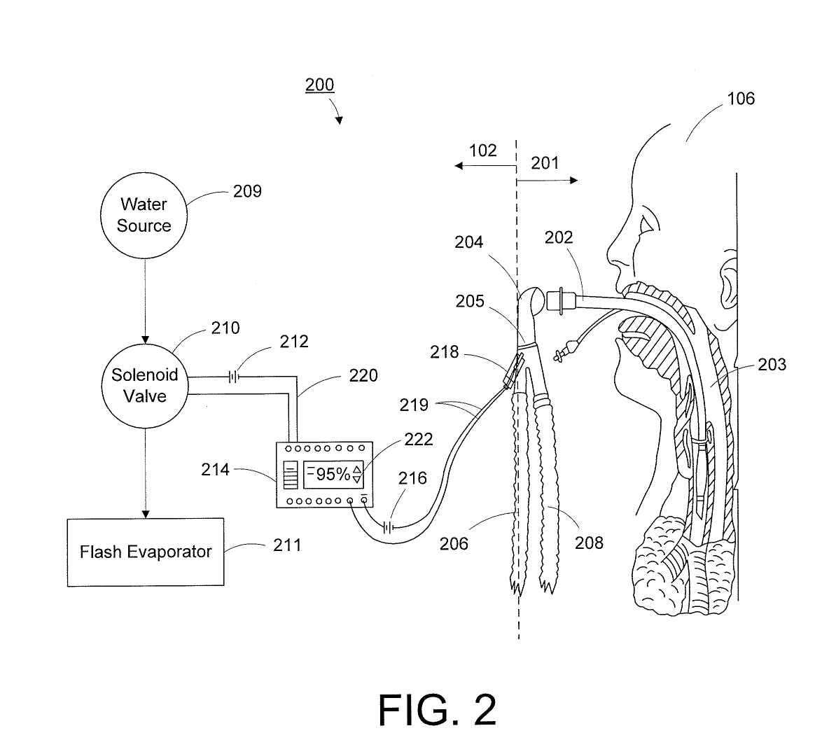

FIG. 2 illustrates a breathing circuit humidification system 200. The breathing circuit humidification system 200 can include the humidification system 102 coupled to a breathing circuit 201. The breathing circuit 201 can be inserted through a trachea 203 of a patient 106, which can be an intubated-neonate, a child, an adult, an animal, and the like.

The breathing circuit 201 can include an inhalation device, such as a tubular device (for example, endotracheal tubular device) 202 that can go inside the mouth of the patient 106 and can pass through the trachea 203 of the patient 206. The tubular device 202 can be connected to a tube 204 that can branch out, via a patient wye “Y” piece device 205, to an inspiratory portion 206 and an expiratory portion 208. In some other implementations, the use of tubular device 204 can be optional such that the patient wye “Y” piece device 205 directly connects to the tubular device 202. The inspiratory portion 206 can allow humidified gas to be provided from the humidification system 102 to the patient 106. To allow this provision of humidified gas by the inspiratory portion 206, the lower (as illustrated) portion of the inspiratory portion is connected to an opening 518 (described below) of a moisture exchanger 502 (described below) that is a part of the humidification system 102. The expiratory portion 208 can facilitate air exhaled by the patient 106 to exit the breathing circuit humidification system 200.

The humidification system 102 can include a water source (for example, a water reservoir) 209, a solenoid valve 210, a flash evaporator 211, a first power source 212, a control circuit 214, a second power source 216, and at least one humidity sensor 218. The humidity sensor 218 can measure humidity (for example, moisture level) of the gas in the inspiratory portion 206. Based on the humidity measured using the humidity sensor 218 and/or based on a recommendation by a clinician (for example, at least one of a cardiologist, an ear-nose-throat specialist, physician, nurse, and the like), the breathing circuit 201 can determine a recommended value or range of humidity (for example, percentage of water vapor in gas/breathing-air) that is recommended to be present in gas (for example, breathing air). The humidity sensor 218 can send a signal indicating the recommended range of the humidity to a control circuit (for example, a computer, a microcontroller, a microprocessor, or the like) 214. This signal can be transmitted via two or more signal wires 219. In one example, the control circuit 214 can be a proportion-integral-derivative (PID) controller that can perform a control loop feedback mechanism. The PID controller 214 can implement a PID routine (for example, a computer program/code) so as to control a relay output 220 in accordance with the recommended input humidity range or point level that can be input automatically (or can be input manually, by a user on a graphic user interface, as described in more detail below). The PID controller 214 can minimize a determined error value by adjusting process control inputs, wherein the error is a difference between a measured process variable (for example, one or more humidity values) and a desired set-point. The relay output 220 can control selectively opening of the solenoid valve 210 in accordance with recommended range or point level of humidity in the gas. The selective opening of the solenoid valve 210 can deliver a controlled amount of water from the water source 209 to the flash evaporator 211. Although use of a humidity sensor 218 is used, other sensors can also be used, such as at least one temperature sensor, at least one pressure sensor, and the like. The temperature sensor can be used to control temperature of gas provided to the patient 106. The pressure sensor can be used to control pressure of gas provided to the patient 106.

Although an automatic input, to the control circuit 214, of the recommended range or one point level of the humidity is described, in some implementations, the control circuit 214 can include a graphic user interface 222 that can receive an input of the range or point level from a user, such as a clinician, a patient 106, an associate of the patient 106, or the like. Although the control circuit 214 is described to include the graphic user interface 222, in some implementations, the control circuit 214 can be connected to a separately (for example, remotely) implemented graphic user interface. The remote implementation of the graphic user interface can be over a network, such as a wired network, wireless network, blue tooth network, infrared network, ZigBee network, local area network, wide area network, metropolitan area network, internet, and the like.

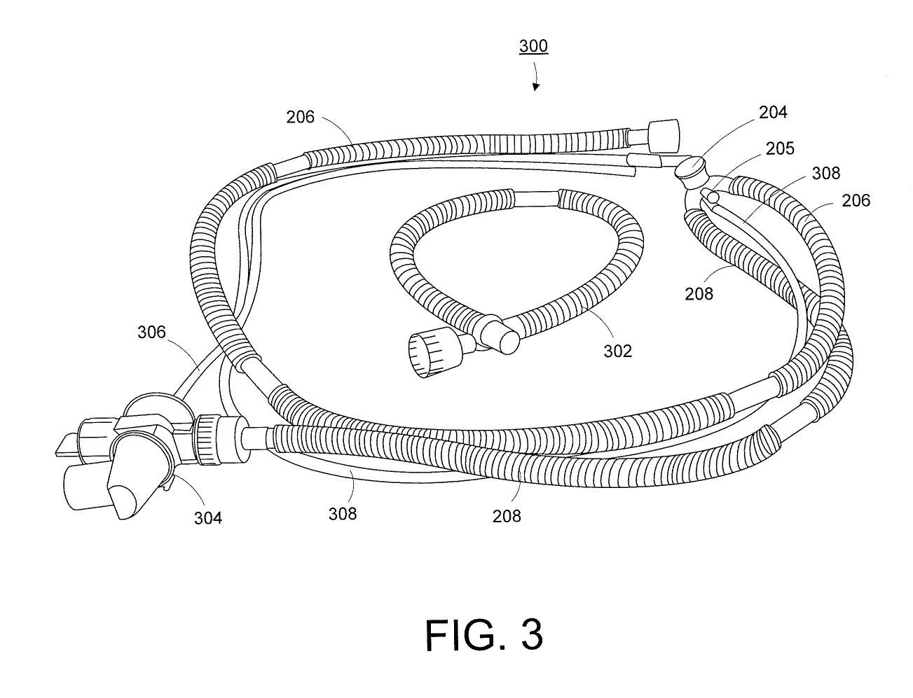

FIG. 3 illustrates a portion 300 of the breathing circuit 201 noted above. The portion 300 includes the tube 204 that can branch, via a patient wye “Y” piece device 205, into an inspiratory portion 206 and an expiratory portion 208. The tube 204 can be connected to the tubular device 202 that can pass through trachea 203 of the patient 106, as described above. Length of at least one of the inspiratory portion 206 and the expiratory portion can be extended by using the extension portion 302. The expiratory portion 208 can be connected to an exhalation valve 304 that can fold or close to prevent return of gas exhaled by patient 106. The exhalation valve 304 can be connected to a trigger tube 306 that can allow the exhaled gas to exit the breathing circuit 201 (described above). The exhalation valve 304 can trigger the pass-out/exit of the exhaled gas via the trigger tube 306. The inspiratory portion 206 can be connected to a patient airway tube 308 that can be further connected to at least one humidity sensor 218 (described above), at least one temperature sensor, and/or at least one pressure sensor. Although the sensors are described as being connected to inspiratory portion 206 via patient airway tube 308, in some other implementations, the sensors can be implemented within the inspiratory portion 206.

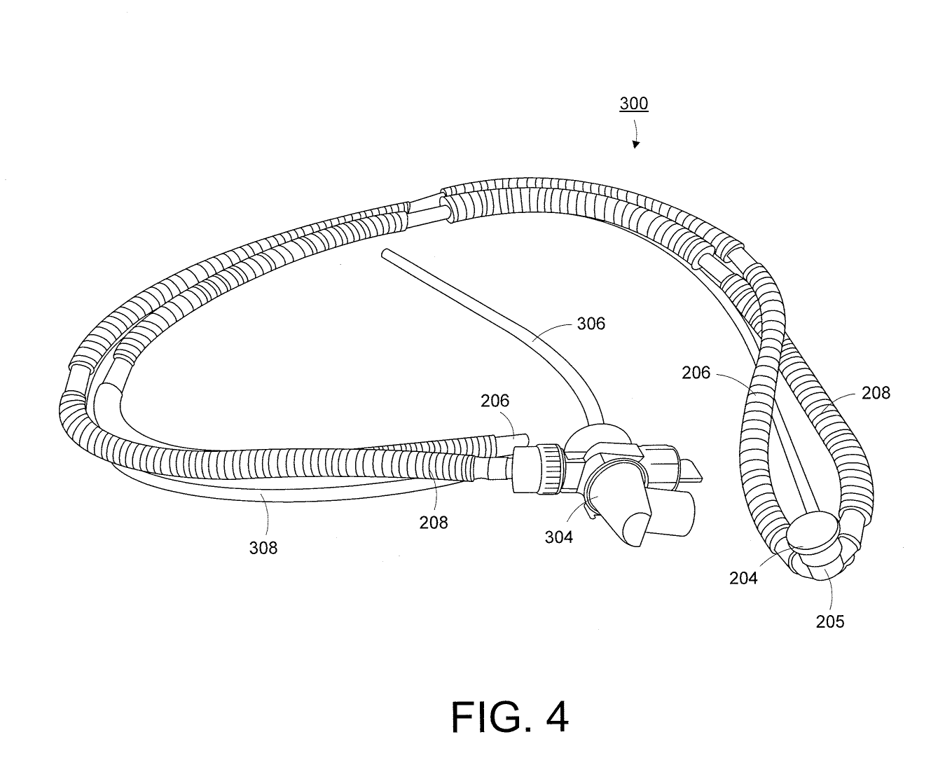

FIG. 4 illustrates another orientation of portion 300 of the breathing circuit 201 noted above. The portion 300 includes the tube 204 that can branch, via a patient wye “Y” piece device 205, into an inspiratory portion 206 and an expiratory portion 208. The tube 204 can be connected to the tubular device 202 that can pass through trachea 203 of the patient 106, as described above. The expiratory portion 208 can be connected to an exhalation valve 304 that can fold or close to prevent return of gas exhaled by patient 106. The exhalation valve 304 can be connected to a trigger tube 306 that can allow the exhaled gas to exit the breathing circuit 201 (described above). The exhalation valve 304 can trigger the pass-out/exit of the exhaled gas via the trigger tube 306. The inspiratory portion 206 can be connected to a patient airway tube 308 that can be further connected to at least one humidity sensor 218 (described above), at least one temperature sensor, and/or at least one pressure sensor.

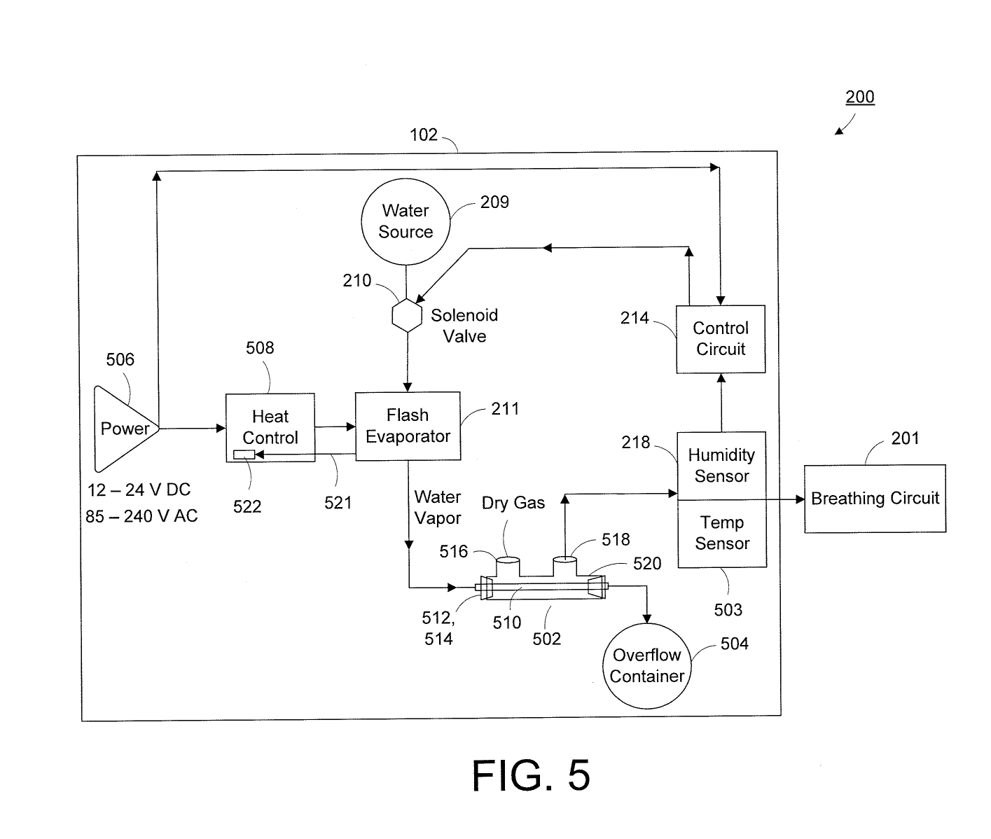

FIG. 5 illustrates a breathing circuit humidification system 200. The breathing circuit humidification system 200 can include the humidification system 102 coupled to a breathing circuit 201. The humidification system 102 can include a water source (for example, a water reservoir) 209, a solenoid valve 210, a flash evaporator 211, a moisture exchanger 502, an overflow collector 504, a control circuit 214, at least one humidity sensor 218 that can be coupled with at least one temperature sensor 503, a power source 506, and a heat control unit 508.

The water source 209 can be a portable water reservoir. Although a portable water reservoir is described, other water sources can also be possible, such as a fixed stationary water reservoir. The water source 209 can provide a continuous supply of water when required. In other implementations, the water source 209 can provide a fixed supply of water, and the water source 209 can be refilled with water. In some implementations, the water source 209 can be refilled with recycled water, which can be water in overflow collector 504 that is recycled and/or purified.

The flash evaporator 211 can selectively receive water from the water source 209. The selective receipt of water can be allowed by selective opening and/or closing of the solenoid valve 210. The water can be provided using a gravitational force. Although gravitational force is described, other implementations can include provision of water by pumping using a pump or by pressurizing. The solenoid valve 210 can be opened and/or closed manually or automatically until sufficient water vapor has been mixed with received gas (for example, dry breathing air, such as dry oxygen). The flash evaporator 211 can include at least one heating element 602 and a tee (T) shaped structure 604 with inlets and outlets, as described in more detail below. At least some portion of the at least one heating element 602 can slide over or into/within a portion of the tee (T) shaped structure 604. The sliding over of some portion of the heating element 602 can be advantageous, as connection of the power source 506 with the heating element 602 can be easier than a possible corresponding connection when the entire heating element 602 slides within the tee (T) shaped structure 604. Although some portion of the at least one heating element 602 is described as sliding over a portion of the tee (T) shaped structure 604, other implementations can also be possible, such as the at least one heating element 602 sliding within a tubular portion of the tee (T) shaped structure 604. The heating element 602 of the flash evaporator 211 can evaporate the received water to form water vapor. Heating of the water to form water vapor by the heating element 602 can be advantageous, as absence of such a heating element 602 can cause water to be inhaled by a patient so as to possibly cause pulmonary edema.

The water vapor can be sent from the flash evaporator 211 to a semi-permeable membrane tube 510 within the moisture exchanger 502. One end 512 of the semi-permeable membrane tube 510 can form the first opening 514 of the moisture exchanger 502. When the water vapor enters the semi-permeable membrane tube 510, some of the water vapor can liquefy to form liquid water. The semi-permeable membrane tube 510 can have a property of allowing water vapor to permeate/escape out through the surface of the semi-permeable membrane tube 510 while prohibiting the liquid water to permeate/escape out through the surface of the semi-permeable membrane tube 510. So, the water vapor can permeate/escape out of the semi-permeable membrane tube 510 while the liquid water may not permeate/escape out of the semi-permeable membrane tube 510. The liquid water may not permeate/escape out of the semi-permeable membrane tube 510 as material forming the semi-permeable membrane tube 510 may not allow liquid to permeate/infuse/pervade through.

A second opening 516 of the moisture exchanger 502 can receive gas from a gas source. The received gas can be dry breathing air, such as dry oxygen. Although dry oxygen is described as the received gas, the received gas can also include a mixture of multiple gases including oxygen, carbon-dioxide, nitrogen, hydrogen, helium, neon, and the like, as present in natural breathing air or as recommended by a clinician. In some implementations, the proportions of gases in the received gas can vary based on a location of the patient 106, because percentage of oxygen and other gases in breathing air can vary based on altitude and other environmental factors, such as industrialization, greenery, and the like. In one implementation, the proportions of gases in the received gas can vary based on physical activity or routine of the patient. The received gas can mix with the water vapor that permeates out of the semi-permeable membrane tube 510 to form humidified gas (for example, humidified oxygen).

Through a third opening 518 of the moisture exchanger 502, the moisture exchanger 502 can transport/send the humidified gas to an inspiratory portion 206 (described above) of the breathing circuit 201. The breathing circuit 201 can control a provision of humidified breathing air to the patient 106.

Through a fourth opening 520 of the moisture exchanger 502, the moisture exchanger 502 can send/remove/dispose liquefied water vapor in the semi-permeable membrane tube 510, end of which can form the fourth opening, to the overflow collector 504. At least some portion of the liquefied water in the overflow collector 504 can be recycled and sent to the water source 209.

The humidity sensor 218 can determine whether an appropriate/recommended amount of water vapor has been mixed with the received gas (for example, dry oxygen). The supply of water vapor to the moisture exchanger 502 can be provided until the appropriate/recommended water vapor has been mixed with the received gas (for example, dry oxygen). For the water vapor supply, water vapor can be produced by the flash evaporator 211. To produce the water vapor, water can be provided to the flash evaporator 211 by the water source 209. To provide the water to the flash evaporator 211, the solenoid valve 210 needs to be open. For the solenoid valve to remain open for receipt of water at the flash evaporator, the control circuit 214 can send an open signal to the solenoid valve 210. Therefore, the solenoid valve 210 can stay, based on a detection of humidity by the humidity sensor 214 and based on an open signal (that is, signal indicating a command to open) by the control circuit 214, open until appropriate/recommended amount of water vapor is mixed with the gas received at second opening 516 of the moisture exchanger 502.

The humidification system 102 can be powered by the power source 506. The power source 506 can be coupled with the heat control unit 508 by a thermocouple 521. The thermocouple 521 can include two different conductors that can produce a voltage proportional to a temperature difference proportional to a temperature difference between one end of each of the two conductors. The power source 506 can be a direct current (DC) power source, such as a battery. As per one example, the direct current (DC) power source can be a 12 Volts DC battery. In one implementation, the battery can be disposable, such as a zinc-carbon battery, an alkaline battery, or the like. In another implementation, the battery can be rechargeable, such as lead-acid battery, automobile battery, rechargeable cell battery, or the like. In some further implementations, the battery can include one or more of galvanic cells, electrolytic cells, fuel cells, flow cells, voltaic piles, or the like. In some examples, the battery can include at least one of a D cell, a C cell, a AA cell, a AAA cell, a AAAA cell, and A23 battery, a 9-volt PP3 battery, a pair of button cells such that the battery provides sufficient power to run the system. Chemical storage devices including various lithium ion chemistries can be used as power source 506 such that the power source generates a power density required for operating the device. Although the power source 506 is described as a direct current (DC) power source, in some other implementations, the power source 506 can be an alternating current power source. The heat control unit 508 can include at least one temperature sensor 522 to ensure that temperature of a heating element 602 of the flash evaporator 211 remains within a predetermined range of temperatures.

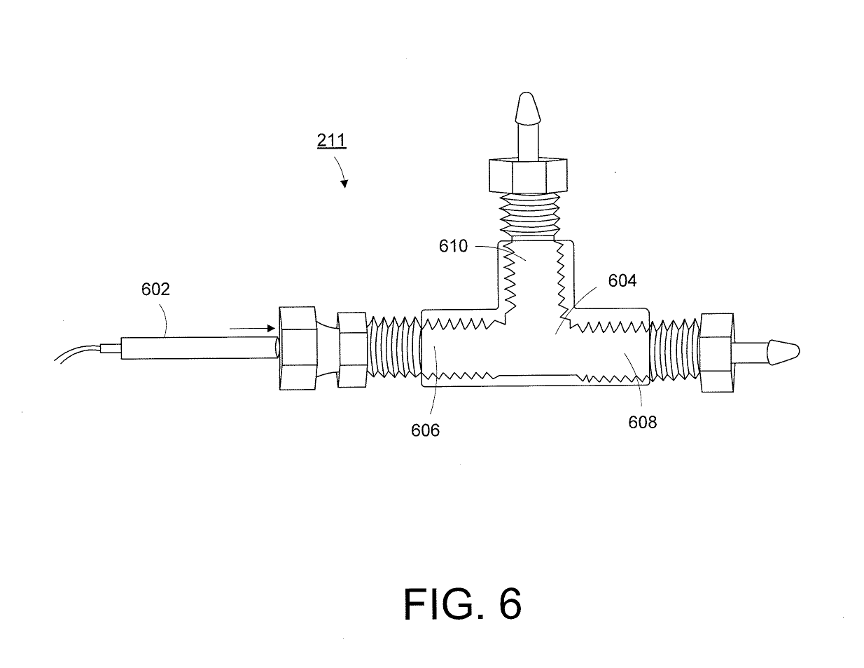

FIG. 6 illustrates a flash evaporator 211. The flash evaporator 211 can include a heating element 602, and a female tee (T) 604. The heating element 602 can be a heating rod with a male port that can couple with the female tee 604 so as to form a compression fitting, thereby sealing the first opening 606. The female tee can include a first opening 606, a second opening 608, and a third opening 610. The heating element 602 can slide into the female tee 604 from the first opening 606 so as to form a compression fitting. The heating element 602 can slide beyond half length of the lateral/longer side of the female tee 604 such that the heating element 602 can heat the entire water received in the flash evaporator 211 to form water vapor. In one example, the heating element 602 can be a tubular heater that can have a diameter of 0.125 inches. The tubular heater can operate using a power of 25-30 watts. This low input power of 25-30 watts for the tubular heater can be provided in portable vehicles, such as automobiles, ambulances, aircrafts, and the like. The tubular heater can mount inside the female tee 604 while some portion, which receives power, can remain external to some portion of the female tee 604, as noted above. The compression fitting can seal around the cylindrical body of heater tube 602.

Water from the water reservoir 209 can selectively enter the flash evaporator 211 from the second opening 608. The second opening 608 can be connected with the solenoid valve 210. The heating element 602 can evaporate the water to form water vapor. The formed water vapor can be passed, through the third opening 610 and by the flash evaporator 211, to the moisture exchanger 502.

The flash evaporator can be enclosed/wrapped by an insulator with very low thermal conductivity, such as mineral wool, KAOWOOL, SUPERWOOL, and/or the like. This enclosing can be further enclosed by an aluminum or carbon-fiber housing/enclosure. Such a construction can ensure that the flash evaporator maintains associated set point temperature with an efficient use of power. The use of an insulator can ensure safety of a user (for example, a patient and/or a clinician) using the humidification system, as the user can be protected from the heat generated internally within the breathing circuit humidification system.

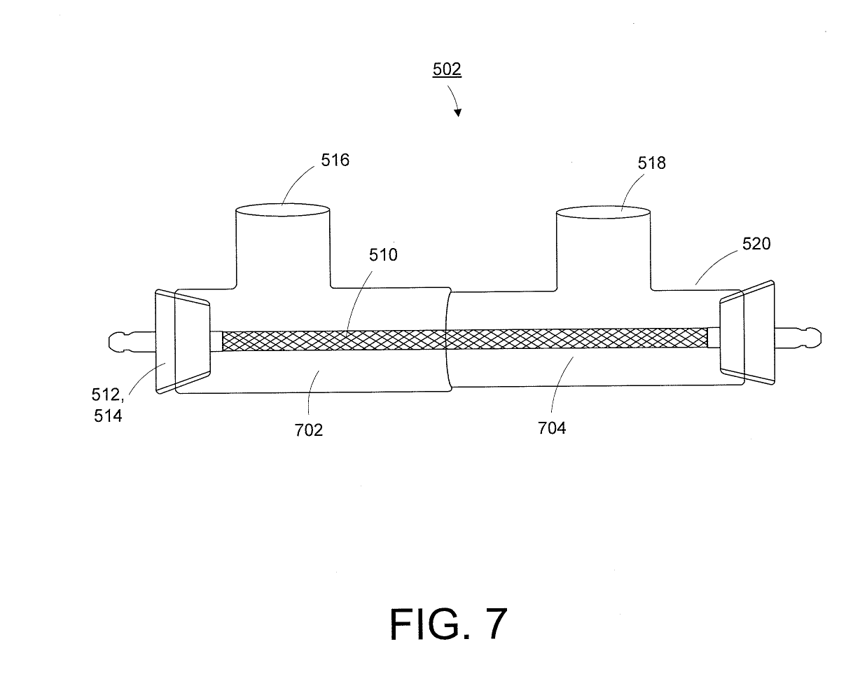

FIG. 7 illustrates a moisture exchanger 502. The moisture exchanger 502 can include tee (T) structure 702 and tee (T) structure 704. The tee (T) structures 702 and 704 can be sealed with each other by forming a sealed joint. The sealed joint can be formed by a sliding over mechanism, a screw mechanism, a tape, soldering, threading, pinning mechanism, or the like. The moisture exchanger 502 can humidify received gas, such as dry breathing air. The semi-permeable membrane tube 510 within the moisture exchanger 502 can receive water vapor from the flash evaporator 211. In one example, the semi-permeable membrane tube 510 can have a length of 6 to 8 inches, an inner diameter of 0.125 inches, or an outer diameter of 0.250 inches.

One end 512 of the semi-permeable membrane tube 510 can form the first opening 514 of the moisture exchanger 502. When the water vapor enters the semi-permeable membrane tube 510, some of the water vapor can liquefy to form liquid water. The semi-permeable membrane tube 510 can have a property of allowing water vapor to permeate/escape out through the surface of the semi-permeable membrane tube 510 while prohibiting (or impeding in some implementations) the liquid water to permeate/escape out through the surface of the semi-permeable membrane tube 510.

A second opening 516 of the moisture exchanger 502 can receive gas from a gas source. The received gas can mix with the water vapor that has permeated out of the semi-permeable membrane tube 510 to form humidified gas.

Through a third opening 518 of the moisture exchanger 502, the moisture exchanger 502 can send the humidified gas to the breathing circuit 201. The breathing circuit 201 can control provision of humidified gas to the patient 106.

Through a fourth opening 520 of the moisture exchanger 502, the moisture exchanger 502 can send/remove/dispose liquefied water vapor in the semi-permeable membrane tube 510, end of which can form the fourth opening, to the overflow collector 504. At least some portion of the liquefied water in the overflow collector 504 can be recycled and sent to the water source 209. The recycling can occur at any or at least one of the overflow collector 504, water source 209, and a recycling body in between the overflow collector 504 and water source 209.

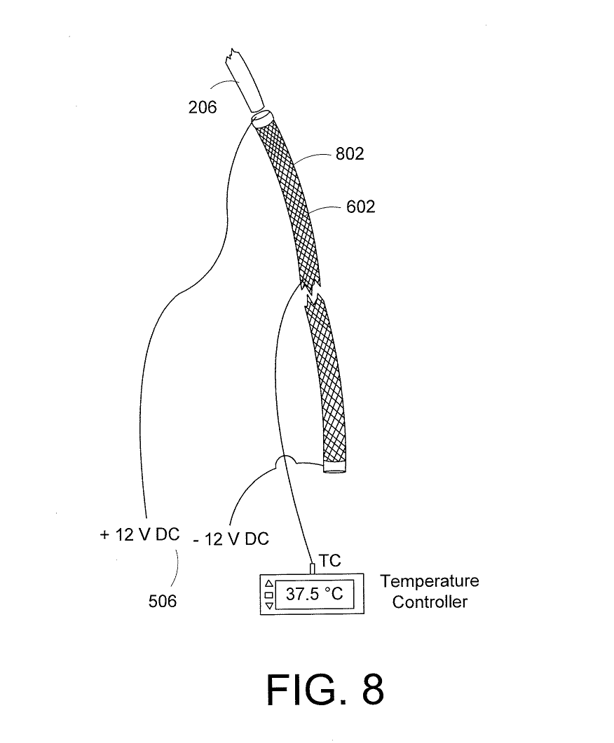

FIG. 8 illustrates a carbon-fiber sleeve (for example, carbon-fiber layer/enclosure/housing) 802 that can enclose/encompass/be-placed-over the inspiratory portion 206 of the breathing circuit 201. The carbon-fiber sleeve 802 can be made of carbon-fiber, which is used as a heater because of electrical resistive property. The electrical resistivity of the carbon fiber can allow maintaining the temperature of the gas in the inspiratory portion 206 above the dew/condensation point. The maintenance of temperature of the gas above the dew/condensation point can prevent condensation from occurring inside the breathing circuit 201, thereby advantageously preventing a patient 106 from breathing in water and possible getting pulmonary edema. The carbon fiber sleeve 802 can be coupled to a controller (for example, temperature controller) 804.

The carbon fiber sleeve 802 can be further coupled to at least one temperature sensor coupled (the coupling can include an adjacent positioning or a remote coupling) to at least one humidity sensor 218 (described above). The output/reading of the at least one temperature sensor can be used to control the temperature in the inspiratory portion 206. The carbon fiber sleeve can be a flexible electrically resistive enclosure that can be created using a vacuum bagging technique that can use a high temperature silicone binder. Although use of high temperature silicone binder is described for vacuum bagging, other techniques can also be possible, such as use of resins. Further, although vacuum bagging is described, other attachment/sealing techniques can also be used, such as molding, carbon molding, filament winding, and the like.

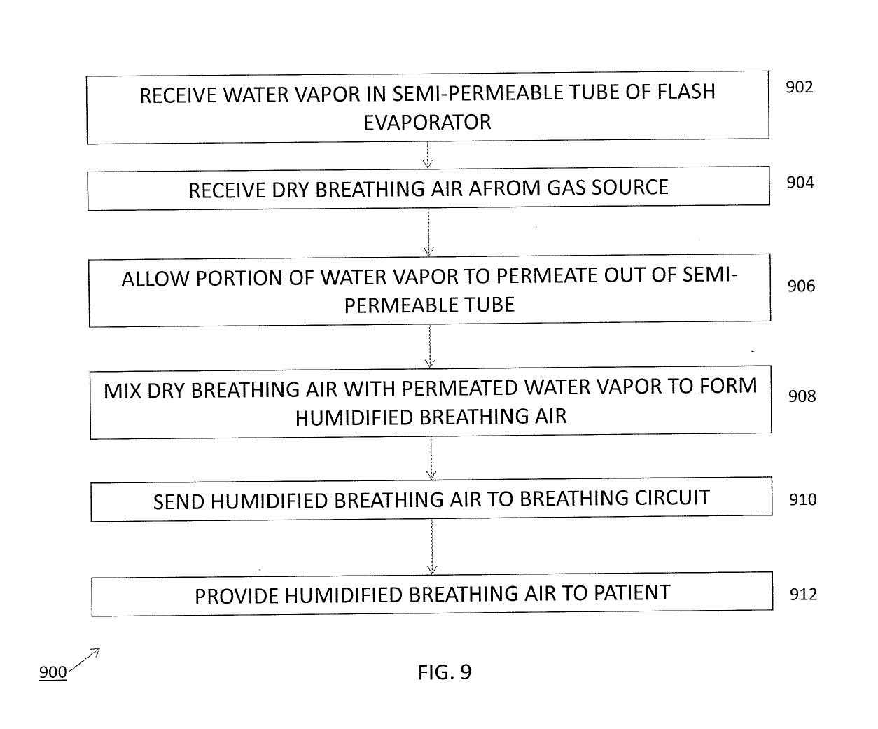

FIG. 9 is a process-flow diagram 900 illustrating humidification of gas using the humidification system. At a first opening 514 in a semi-permeable membrane tube 510 within a moisture exchanger apparatus 502, water vapor can be received, at 902, from a flash evaporator 211. The water vapor can be formed by evaporation of water received at the flash evaporator 211 from a water source 209. A portion of the received water vapor can escape out of the semi-permeable membrane tube 510. At a second opening 516 of the moisture exchanger apparatus 502, gas can be received, at 904, from a gas source. A portion of the water vapor received at 902 can permeate/escape out of the semi-permeable membrane tube 510 at 906. The escaped water vapor can mix, at 908, with the received gas to form humidified gas. The humidified gas can be transported/sent, at 910, to the breathing circuit 201. The humidified gas at the breathing circuit 201 can be provided, at 912, in a controlled manner to the patient 106.

Some components of the humidification system 102 can be disposable. For example, water source 209, moisture exchanger 502 (inclusive of components 510, 512, 514, 516, 518, 520), and overflow container 210 can be disposable. Further, some components of the breathing circuit 201 can be disposable, such as components 202, 204, 206, and 208. Such components can include, by a direct or indirect contact with a patient, some portion of saliva and/or air exhaled by the patient 106. To prevent transmission of infection, microorganisms, and/or the like, such components can be disposable as such a transmission can cause cross-contamination between various patients. Further, wet surface of the moisture exchanger 502 can harbor microorganisms that can cause contamination, if reused. Therefore, the moisture exchanger being disposable can be advantageous for this additional reason.

Some components of the humidification system 102 can be reusable. For example, power source 506, heat control unit 508, flash evaporator 211, solenoid valve 210, and control circuit 214 can be reusable. Reuse of such components can be advantageously cost efficient, thereby saving cost for a patient.

Aspects of the subject matter described herein may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor) for displaying information to the user and a keyboard and a pointing device (e.g., a mouse or a trackball) by which the user may provide input to the computer. Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

The subject matter described herein may be implemented in a computing system that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system may include clients and servers. A client and server are generally remote from each other and typically interact through a communication network. The relationship of client and server arises by virtue of computer programs running on the respective computers and having a client-server relationship to each other.

The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1.-38. (canceled)

39. A system comprising:
a water source;
a flash evaporator that selectively receives water from the water source, the flash evaporator evaporating at least a portion of the received water to form water vapor; and
a moisture exchanger that receives the water vapor from the flash evaporator, the moisture exchanger receiving gas from a gas source, the moisture exchanger humidifying the received gas with the received water vapor and transporting the humidified gas to a patient breathing circuit.

40. The system of claim 39, further comprising:
at least one temperature sensor that measures temperature of the humidified gas in the patient breathing circuit; and
at least one humidity sensor that measures humidity of the humidified gas in the patient breathing circuit.

41. The system of claim 40 further comprising:
a control unit coupled to the at least one temperature sensor and the at least one humidity sensor, the control unit selectively introducing water from the water source into the flash evaporator, the selective introduction based on the humidity measured by the at least one humidity sensor.

42. The system of claim 39 further comprising:
a solenoid valve that selectively opens to selectively introduce water from the water source and to the flash evaporator, the selective introduction of water causing the selective receiving of the water by the flash evaporator;
a control unit that controls, based on an amount of moisture present in the patient breathing circuit, the selectively opening of the solenoid valve.

43. The system of claim 42, wherein the water source provides a continuous supply of water that selectively passes through the solenoid valve.

44. The system of claim 43, wherein the continuous supply of water is provided using a gravitational force.

45. The system of claim 42 further comprising:
a temperature sensor that determines a temperature of the humidified gas; and
a humidity sensor that determines the amount of moisture present in the patient breathing circuit.

46. The system of claim 45, wherein:
the flash evaporator comprises a heating element that evaporates the water to form the water vapor; and
the heating element temporarily stops evaporating the water when the temperature determined by the temperature sensor exceeds a predetermined threshold.

47. The system of claim 45, wherein an amount of the water vapor and an amount of the received gas that are received at the moisture exchanger are based on the amount of moisture determined by the humidity sensor.

48. The system of claim 39 further comprising:
an overflow collector that collects excess water vapor that is not mixed with the received gas to form humidified gas.

49. The system of claim 42 further comprising:
a direct current voltage source that provides power to at least one of the flash evaporator, the moisture exchanger, and the control unit.

50. The system of claim 42 further comprising:
an alternating current voltage source that provides power to at least one of the flash evaporator, the moisture exchanger, and the control unit.

51. The system of claim 39, wherein:
the flash evaporator comprises a structure having a tee (T) shape, the structure having a first gap, a second gap, and a third gap; and
the flash evaporator comprises a heating element that evaporates water to form water vapor, the heating element coupled to the tee (T) shaped structure so as to seal the first gap.

52. The system of claim 51, wherein:
the water from the water source is received by the flash evaporator from the second gap; and
the third gap is sealed by a fixed solenoid valve.

53. The system of claim 51, wherein the heating element slides along an inside surface of a portion of the tee (T) shaped structure.

54. The system of claim 51, wherein the heating element slides along an outside surface of a portion of the tee (T) shaped structure.

55. The system of claim 39, wherein the moisture exchanger comprises a semi-permeable membrane tube that receives the water vapor from the flash evaporator, the semi-permeable membrane tube allowing a first portion of the received water vapor to escape out of the semi-permeable membrane tube while prohibiting a second portion of the received water vapor that condenses to form liquid water to escape out of the semi-permeable membrane tube.

56. The system of claim 55, wherein the water vapor that escapes out of the semi-permeable membrane tube mixes with the gas received at the moisture exchanger to form humidified gas that is sent to the patient breathing circuit.

57. The system of claim 39, wherein the patient breathing circuit and the moisture exchanger are disposable while at least the water source and the flash evaporator are reusable.

58. The system of claim 39, wherein the received gas comprises a breathing gas including at least one of oxygen, carbon dioxide, nitrogen, helium, and neon.

59. The system of claim 39, wherein:
the patient breathing circuit comprises an inspiratory portion and an expiratory portion; and
the inspiratory portion is enclosed by a carbon-fiber enclosure.
